(19) United States
(12) Patent Application Publication
(10) Pub. No.: US 2026/0167793 A1
(43) Pub. Date: Jun. 18, 2026

(54) PROCESSING OF EXPANDED POLYETHYLENE ABOVE THE MELT POINT

(71) Applicant: W. L. Gore & Associates, Inc., Newark, DE (US)

(72) Inventors: Jeffrey B. Duncan, Flagstaff, AZ (US); Thomas R. McDaniel, Flagstaff, AZ (US)

(21) Appl. No.: 19/139,566

(22) PCT Filed: Dec. 15, 2023
(86) PCT No.: PCT/US2023/084361

§ 371 Date: Jun. 16, 2025

Related U.S. Application Data
(60) Provisional application No. 63/433,114, filed on Dec. 16, 2022.

(51) Int. Cl. C08J 9/36; A61L 27/16; C08J 5/12

(52) U.S. Cl. CPC C08J 9/36; A61L 27/16; C08J 5/121; C08J 2323/06

(57) ABSTRACT

A method of processing expanded polyethylene (ePE) comprising positioning an ePE substrate onto a heated component, the ePE substrate having a first size as the ePE substrate is heated on the heated component, removing the ePE substrate from the heated component such that the ePE substrate cools and retracts to second size, wherein the second size is smaller than the first size, and forming the ePE substrate into an ePE article.

### CROSS-REFERENCE TO RELATED APPLICATION

This application is a national phase application of PCT Application No. PCT/US 2023/084361, internationally filed on Dec. 15, 2023, which claims the benefit of Provisional Application No. 63/433,114, filed Dec. 16, 2022, which are incorporated herein by reference in their entireties for all purposes.

### FIELD

The present disclosure relates generally to apparatuses, systems, and methods for processing expanded polyethylene (ePE). More specifically, the disclosure relates to apparatuses, systems, and methods that include processing expanded polyethylene (ePE) that may be used in medical devices.

### BACKGROUND

Methods used for processing materials are important as the method can impart specific qualities onto the processed materials. The specific qualities may be necessary for the processed material to function for its intended purpose or may allow the processed materials to be used in new ways. Selection of processing methods is important in a variety of industries, including, but not limited to the medical device industry, and more specifically for implantable medical devices. However, processed materials may be used across various industries and the same properties that are desirable in one industry may also be important in other industries.

Medical devices often need to be adaptable to fit the needs of a patient. For example, implantable devices made of processed materials may need to fit within a length or a diameter of a lumen of the patient. However, it is costly to make and store implantable devices in a various sizes in small increments. Implantable devices may be provided only at sizes in specific intervals that are determined by average sizes and then used based on closest fit. However, closest fit does not necessarily mean closest fit. Furthermore, lumen diameters may be different along a length of the patient's lumen, and therefore may be difficult to determine the appropriate size for an implantable device. What is needed are materials that are useful for providing medical devices that can provide optimal fits when implanted.

### SUMMARY

The present disclosure relates to methods and articles produced by such methods for processing ePE. For example, methods and articles produced by such methods include exposing ePE to temperatures above the melt temperature in processing, which are above the melt point of ePE, ePE exhibits desirable characteristics. Such desirable characteristics may include diameter adjustability, improved adhesion to metals, improved adhesion to other polyethylene, ability to store length, ability to distend, and abrasion resistance.

According to one example (“Example 1”), a method of processing expanded polyethylene (ePE) comprises positioning an ePE substrate onto a heated component, the ePE substrate having a first size, heating the ePE substrate on the heated component, removing the ePE substrate from the heated component such that the ePE substrate cools and retracts to second size, wherein the second size is smaller than the first size, and forming the ePE substrate into an ePE article.

According to another example (“Example 2”), further to Example 1, forming the ePE substrate into the ePE article includes wrapping the ePE substrate onto a mandrel.

According to another example (“Example 3”), further to Example 2, forming the ePE substrate into the ePE article includes melt bonding the ePE substrate to itself along a longitudinal line.

According to another example (“Example 4”), further to Example 1, positioning the ePE substrate onto the heated component includes providing the ePE substrate as a sheet of ePE.

According to another example (“Example 5”), further to Example 1, heating the ePE substrate on the heated component includes heating the heated component between about 110 degrees Celsius and 180 degrees Celsius.

According to another example (“Example 6”), further to Example 1, the method comprises adhering the ePE article to a metal.

According to another example (“Example 7”), further to Example 1, the method further comprises adhering the ePE article to another polyethylene structure.

According to another example (“Example 8”), further to Example 1, the method further comprises distending at least a portion of the ePE article.

According to another example (“Example 9”), further to Example 8, distending at least a portion of the ePE article includes radial expansion.

According to another example (“Example 10”), further to Example 8, distending at least a portion of the ePE article includes longitudinal expansion.

According to one example (“Example 11”), a method of processing expanded polyethylene (ePE) comprises heating an ePE substrate to a temperature above a melt temperature of ePE, the ePE substrate having a first size, cooling the ePE substrate, the ePE substrate retracting to a second size upon cooling, the second size being smaller than the first size, and forming the ePE substrate into an ePE article.

According to another example (“Example 12”), further to Example 11, the ePE substrate is heated between about 110 degrees Celsius and 180 degrees Celsius.

According to another example (“Example 13”), further to Example 11, the method further comprises the step of distending at least a portion of the ePE article.

According to another example (“Example 14”), further to Example 13, distending at least a portion of the ePE article is done at a room temperature.

According to one example (“Example 15”), an ePE article made of expanded polyethylene (ePE) comprises an ePE substrate having been formed into an ePE article, the ePE article being formed by retracting the ePE sheet via a heating to cooling process, the ePE article being capable of distention.

According to another example (“Example 16”), further to Example 15, the ePE substrate is formed into a graft.

According to another example (“Example 17”), further to Example 15, the ePE substrate is formed into the ePE article using a mandrel.

According to another example (“Example 18”), further to Example 15, the ePE article is capable of distention in a longitudinal direction.

According to another example (“Example 19”), further to Example 15, the ePE article is capable of distention in a radial direction.

According to another example (“Example 20”), further to Example 15, the ePE article is porous.

The foregoing Embodiments are just that, and should not be read to limit or otherwise narrow the scope of any of the inventive concepts otherwise provided by the instant disclosure. While multiple embodiments are disclosed, still other embodiments will become apparent to those skilled in the art from the following detailed description, which shows and describes illustrative embodiments. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is a block diagram of a method of processing expanded polyethylene (ePE) including a positioning step, a removing step, and a forming step, in accordance with some embodiments;

FIG. 2 is a block diagram of the method of processing expanded polyethylene (ePE) of FIG. 1 further including a distending step, in accordance with some embodiments;

FIG. 3 is a block diagram of a method of processing expanded polyethylene (ePE) including a heating step, a cooling step, and a forming step, in accordance with some embodiments;

FIG. 4 is a block diagram of the method of processing expanded polyethylene (ePE) of FIG. 3 further including a distending step, in accordance with some embodiments;

FIG. 5 illustrates an example in which a tubular article is formed from an ePE sheet, in accordance with some embodiments;

FIG. 6 illustrates an example in which a flat article is formed from an ePE sheet, in accordance with some embodiments;

FIG. 7 illustrates an embodiment in which the tubular article of FIG. 5 is distended to an intermediate size, is accordance with some embodiments;

FIG. 8 illustrates an embodiment in which the flat article of FIG. 6 is distended to an intermediate size, in accordance with some embodiments;

FIGS. 9A and 9B illustrate a microstructure of ePE substrates of Example 1 processed above the melt temperature;

FIG. 10 illustrates thickness data for ePE substrates of Examples 1 and 2;

FIG. 11 illustrates bubble point data for ePE substrates of Examples 1 and 2;

FIG. 12 illustrates air leak data for ePE substrates of Examples 1 and 2; and

FIG. 13 illustrates peel data for ePE substrates of Example 2.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

The term “laminate” as used herein refers to multiple layers of membrane, composite material, or other materials, such as, but not limited to a polymer, such as, but not limited to an elastomer, elastomeric or non-elastomeric material, and combinations thereof.

The term “film” as used herein generically refers to one or more of the membrane, composite material, or laminate.

The term “biocompatible material” as used herein generically refers to any material with biocompatible characteristics including synthetic materials, such as, but not limited to, a biocompatible polymer, or a biological material, such as, but not limited to, bovine pericardium. Biocompatible material may comprise a first film and a second film as described herein for various embodiments.

The term “polyethylene” (PE) as used herein is inclusive of all types of polyethylene, including but not limited to expanded polyethylene (ePE).

### Description of Various Embodiments

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

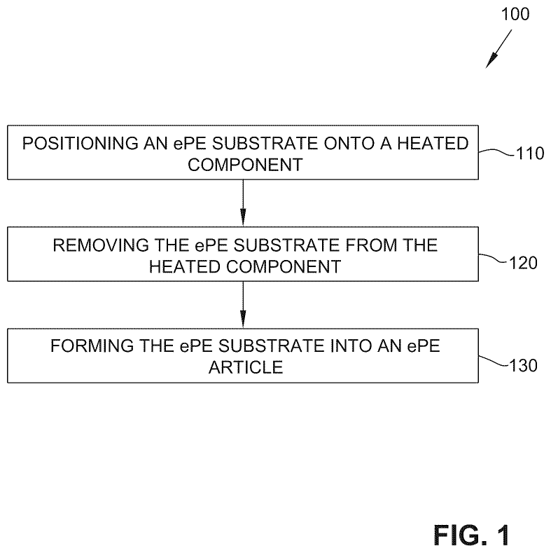

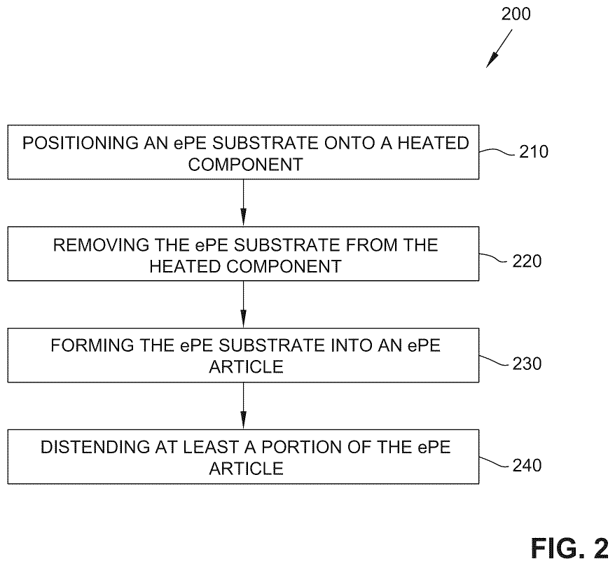

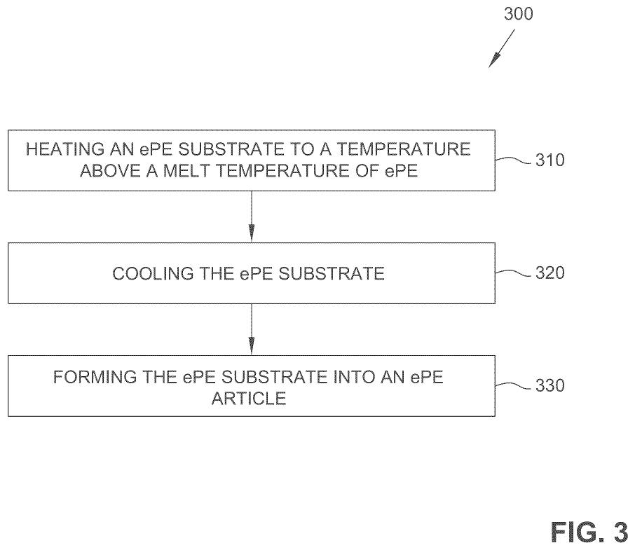

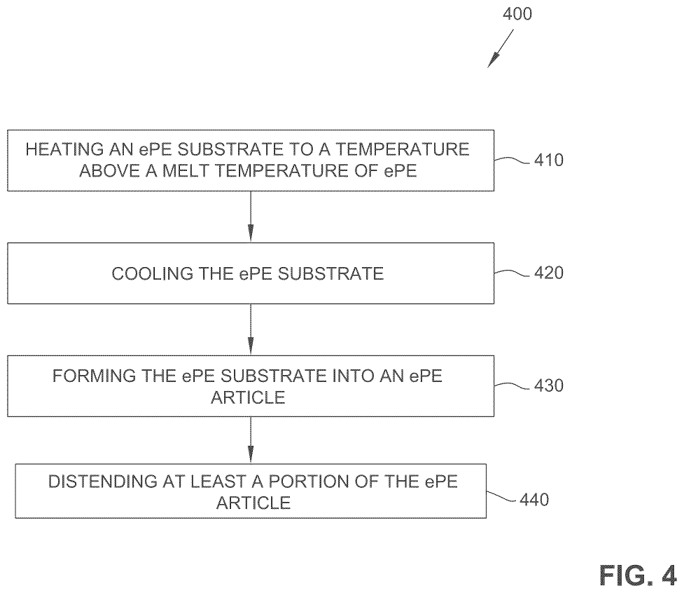

The method shown in FIGS. 1 and 2 is provided as an example of the various features of the method and, although the combination of those illustrated features is clearly within the scope of invention, the embodiments and their illustrations are not meant to suggest the inventive concepts provided herein are limited from fewer features, additional features, or alternative features to one or more of those features shown in FIGS. 3 and 4. For example, in various embodiments, the process steps of FIGS. 1 and 2 may include the process steps described with reference to FIGS. 3 and 4. It should also be understood that the reverse is true as well. One or more of the components depicted in FIGS. 3 and 4 can be employed in addition to, or as an alternative to components depicted in FIGS. 1 and 2. For example, the method steps of the method shown in FIGS. 3 and 4 may be employed in connection with the method steps of the method shown in FIGS. 1 and 2.

FIG. 1 is a block diagram of a method 100 of processing expanded polyethylene (ePE), in accordance with some embodiments. The method 100 can be implemented in a variety of contexts, including but not limited to medical devices, which may include implantable medical devices. Various forms of ePE may be implemented in the methods, including but not limited to membranes, films, tapes, tubes, and so forth. It is further understood that the ePE may be provided with various characteristics including different thicknesses, fibril and node structures, porosity, densities, and so forth. Accordingly, the embodiments discussed herein are not to be limited to specific initial conditions or forms but are understood to broadly understood to incorporate any ePE starting material that is suitable for the described methods.

In some embodiments, the method of processing ePE 100 positioning an ePE substrate onto a heated component 110, removing the ePE substrate from the heated component 120 and forming the ePE substrate into an ePE article 130.

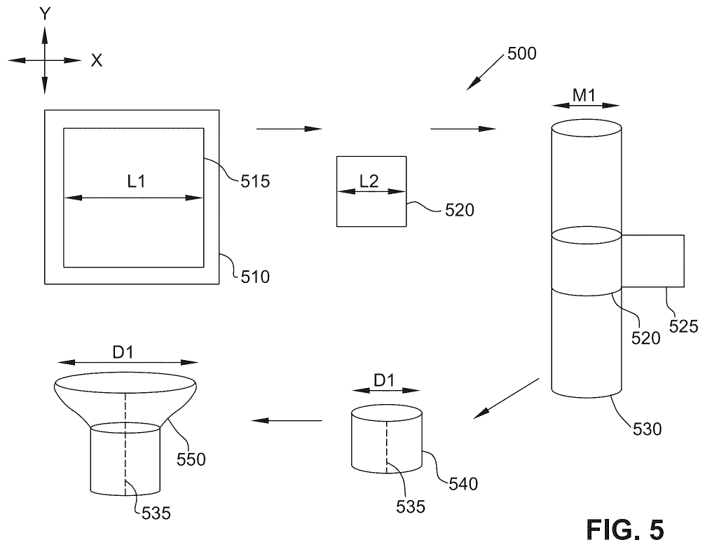

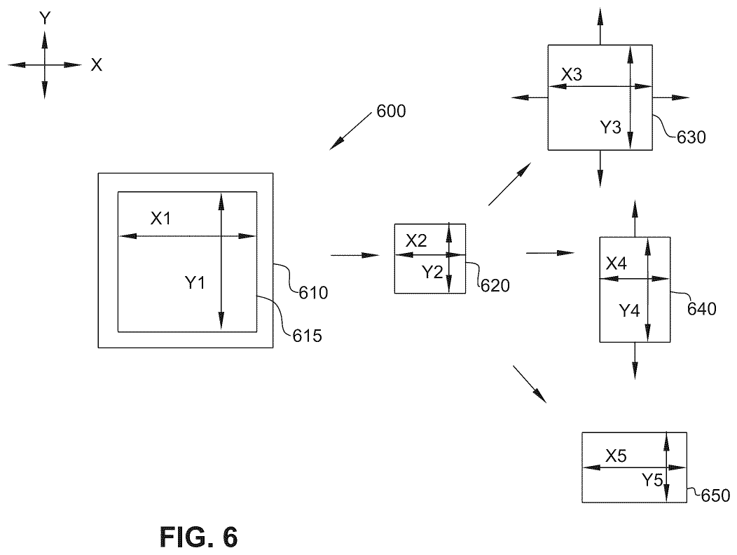

In some embodiments, when the ePE substrate is positioned on the heated component, for example the heated component 510, 610 as seen in FIGS. 5-6, heat is transferred from the heated component to the ePE substrate such that the ePE substrate is heated by the heated component. Although not limited to these embodiments, the heated component may include a press, a heated pad, an oven, or the like. The heated component may be provided at various temperatures, including but not limited to, above a melt temperature of ePE. For example, the temperature of the heated component may be provided between about 110° C. to about 180° C. In some embodiments, the heated component is provided at a temperature of from about 110° C. to about 120° C., from about 120° C. to about 130° C., from about 130° C. to about 140° C., from about 140° C. to about 150° C., from about 150° C. to about 160° C., from about 160° C. to about 170° C., and from about 170° C. to about 180° C. The heated component may be provided at any appropriate temperature prior to positioning the ePE substrate onto the heated component 110, or the heated component may be heated to the appropriate temperature after positioning the ePE substrate onto the heated component 110. In some embodiments, when positioning the ePE substrate onto the heated component 110 and the heated component is at or above the melt temperature of ePE, the ePE substrate may be heated on the heated component to a temperature above the melt temperature of ePE. In some embodiments, the ePE substrate is heated unconstrained. In other embodiments, the ePE substrate may be constrained in an X-direction (e.g., horizontally), a Y-direction (e.g., longitudinally), or in both the X-direction and the Y-direction. In some embodiments, positioning the ePE substrate onto the heated component 110 may also include providing the ePE substrate as a sheet of ePE. The sheet of ePE has a first size as the ePE substrate is heated on the heated component. The sheet of ePE may be provided as a square, a rectangle, or other shapes. In one embodiment, the sheet of ePE may be the square (see FIG. 5) and the first size may be defined by a first dimension L1 (see FIG. 5).

Referring still to FIG. 1, the method further includes removing the ePE substrate from the heated component 120. For example, after a specified amount of time, after a target ePE substrate temperature is reached, or when desired material properties are achieved, the ePE substrate is removed from the heated component. In removing the ePE substrate from the heated component 120, the ePE substrate is removed from the heated component such that the ePE substrate cools. The ePE substrate may be cooled at room temperature, may be placed in an environment that is cooler than room temperature (e.g., a freezer), or may be slowly cooled in an environment with a temperature higher than room temperature. In some embodiments, the environment in which the ePE substrate is cooled may be at a stable temperature or may be a variable temperature. In some embodiments, the variable temperature of the environment allows the ePE substrate to be cooled at a controlled rate. The rate of cooling of the ePE substrate may be constant or may be variable.

As the ePE substrate cools, the ePE substrate retracts to a second size. The second size may be smaller than the first size. Continuing the embodiment where the sheet of ePE is the square sheet (e.g., as shown in FIG. 5), the second size may be defined by a second dimension L2 (see FIG. 5). The second dimension L2 (FIG. 5) may be smaller than the first dimension L1 (FIG. 5). Although the embodiment of FIG. 5 is referenced here, a rectangular sheet of ePE (e.g., as shown in FIG. 6) may be subjected to a similar process and would likewise experience a similar change in size upon cooling. The embodiment of the rectangular sheet of ePE is discussed further with respect to FIG. 6. It is understood that once the ePE substrate is cooled such that the ePE is at or below a predetermined temperature, that the retraction of the ePE substrate ceases and the size of the ePE substrate is stable. In some embodiments, the predetermined temperature may be the melt temperature of the ePE substrate. The predetermined temperature may be at least partly dependent on the type of ePE used (e.g., expanded lower molecular weight polyethylene or expanded high molecular weight polyethylene). Once cooled, the ePE substrate is capable of expansion or distention at temperatures substantially below the melt temperature (e.g., room temperature). The ePE substrate may also still capable of expansion or distention at temperatures above the melt temperature.

It is understood that during the cooling process, as the ePE substrate retracts, specific material properties may be imparted to the ePE substrate that may be desirable in certain contexts. For example, the ePE substrate that has been cooled and retracted may be capable of selective expansion or distension, better adhesion to secondary structures such as metals, PE, or other polymers, stored length in the ePE substrate, distension when yielded, and so forth. Similar properties may be imparted when the ePE substrate is expanded or distended after cooling.

Referring still to FIG. 1, after removing the ePE substrate from the heated component 120, the ePE substrate is formed into an ePE article. Forming the ePE substrate into the ePE article 130 may, for example, result in forming an ePE article 540 or forming an ePE article 620 of FIGS. 5-6. Forming the ePE substrate into the ePE article 130 may be done at an elevated temperature at or below the temperature of the heated component. In some embodiments, the ePE article may be formed into a tubular article (e.g., a tubular article 540 of FIG. 5), formed into a flat article (e.g., a flat article 620, 630, 640, or 650 of FIG. 6), formed into a film, or formed into a laminate made of multiple layers of processed ePE. Other articles may also be contemplated. In a medical application, the tubular article may be a graft and the flat article may be a hernia patch, a cardiovascular patch, a neuro membrane, and so forth.

In some embodiments, forming the ePE substrate into the ePE article 130 may further include wrapping the ePE substrate onto a mandrel (e.g., a mandrel 530 of FIG. 5). This may form the ePE substrate into the tubular article (e.g., the tubular article 540 of FIG. 5). Further to this embodiment, forming the ePE substrate into the ePE article 130 may further include bonding or attaching (e.g., bonding using adhesive, melt bonding, or mechanical attachment using a suture) the ePE substrate to itself. In one embodiment, the bonding or attaching of the ePE substrate to itself may be done along a longitudinal line (e.g., a longitudinal melt bond line 535 of FIG. 5). Other embodiments in which the bonding or attaching the ePE substrate to itself is done along a horizontal line, a diagonal line, within a zone, or the like. Bonding or attaching the sheet of ePE to itself may be done when the ePE is wrapped onto the mandrel.

In some embodiments, the method of processing ePE 100 may further include adhering the ePE article to a metal or a metal secondary structure. Adhering the ePE article to the metal or metal secondary structure may be done at the same time as forming the ePE substrate into the ePE article 130 or may be done afterwards. The metal or the metal secondary structure may include a stent, an occluder, a shunt, a valve frame, or the like.

In some embodiments, the method of processing ePE 100 may further include adhering the ePE article to a polyethylene or a polyethylene secondary structure. Adhering the ePE article to the polyethylene or the polyethylene secondary structure may be done at the same time as forming the ePE substrate into the ePE article 130 or may be done afterwards. The polyethylene or polyethylene secondary structure may include a graft, a leaflet, or the like.

Further, in some embodiments, the method of processing ePE 100 may further include adhering the ePE article to a polymer or a polymer secondary structure. The polymer may include polytetrafluoroethylene (PTFE), including, but not limited to expanded polytetrafluoroethylene (ePTFE). Other polymer types may be contemplated.

Referring now to FIG. 2, a block diagram of a method of processing expanded polyethylene (ePE) 200 is provided, in accordance with some embodiments. The method 200 can be implemented in a variety of contexts, including but not limited to medical devices, which may include implantable medical devices.

The method of processing ePE 200 includes positioning an ePE substrate onto a heated component 210, removing the ePE substrate from the heated component 220, forming the ePE substrate into an ePE article 230, and distending at least a portion of the ePE article 240.

The method step of positioning the ePE substrate onto the heated component 210 may be substantially similar to the positioning of the ePE substrate onto the heated component 110 as described above with respect to FIG. 1. The heated component, for example, may be the heated component 510, 610 as seen in FIGS. 5-6. The method step of removing the ePE substrate from the heated component 220 may be substantially similar to the removing the ePE substrate from the heated component 120 as described above with respect to FIG. 1. The method step of forming the ePE substrate into the ePE article 230 may be substantially similar to the forming the ePE substrate into the ePE article 130 as described above with respect to FIG. 1. Forming the ePE substrate into the ePE article, may for example, include the ePE article 540 and/or the ePE article 620 of FIGS. 5-6.

In some embodiments, after retracting the ePE substrate, heat and/or pressure may be applied to the ePE substrate to limit the amount of distension that is possible. This allows the ePE substrate to have a locked-in or designated amount of possible distention. This may allow for control over the size of an ePE article formed from the ePE substrate.

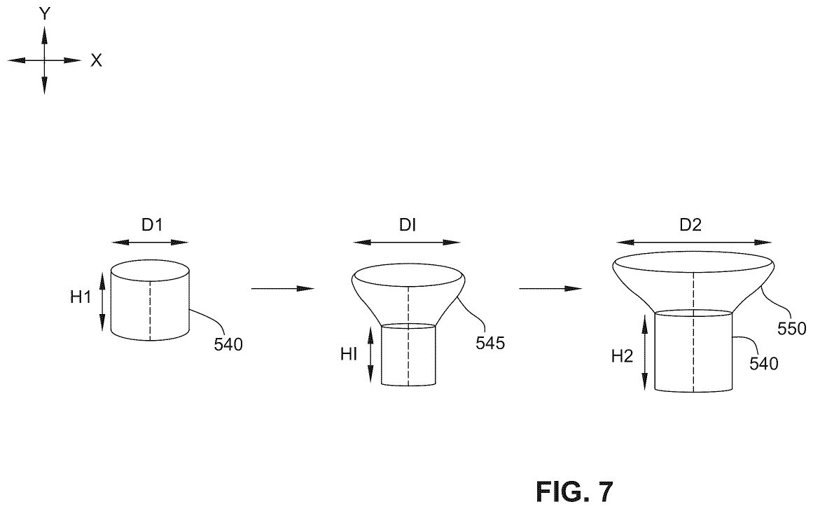

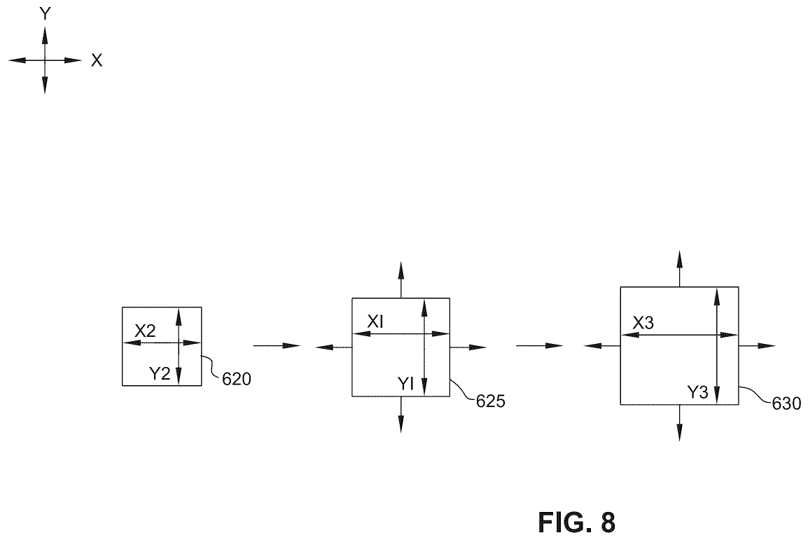

In forming the ePE substrate into an ePE article 230, the ePE article is capable of distention. In some embodiments, distending at least a portion of the ePE article 240 is performed on at least a position of the ePE article. In other embodiments, distending at least a portion of the ePE article 240 may be performed on the entire ePE article. In some embodiments, when the ePE article is at the second size, distending at least a portion of the ePE article 240 may expand the ePE article back to the first size. In some embodiments, when the ePE article is at the second size, distending at least a portion of the ePE article 240 may expand the ePE article to an intermediate size between the first size and the second size. In some embodiments, the intermediate size may include an intermediate diameter, for example an intermediate diameter DI in FIG. 7. In some embodiments, the intermediate size may also include an intermediate length, for example XI and YI in FIG. 8. In other embodiments, the intermediate size may include both the intermediate diameter and the intermediate length, for example the intermediate diameter DI and an intermediate length HI in FIG. 7.

In some embodiments, distending at least a portion of the ePE article 240 includes radial expansion. In some embodiments, distending at least a portion of the ePE article 240 includes one or both of longitudinal expansion and horizontal expansion. The longitudinal expansion may be in a Y-direction. The horizontal expansion may be in an X-direction. In some embodiments, distending at least a portion of the ePE article 240 includes both radial expansion and one or both of longitudinal expansion and horizontal expansion. Other embodiments in which distending at least a portion of the ePE article is done along in a diagonal direction or another non-straight direction are also contemplated.

In some embodiments, distending at least a portion of the ePE article 240 may be done when the ePE article is at room temperature (e.g., around 25° C.-30° C.).

In some embodiments, distension can be done using a balloon (e.g., an angioplasty balloon). In some embodiments, the amount of distension of the ePE article is limited such that distention with the balloon is done to a predetermined ePE article size. In some embodiments, distending at least a portion of the ePE article 240 is done by a manufacturer of the ePE article. In some embodiments, distending at least a portion of the ePE article 240 may be performed by a user or another party besides the manufacturer. In some embodiments, distending 240 the ePE article is done by a physician prior to, during, or after a surgical procedure.

FIG. 3 is a block diagram of a method of processing expanded polyethylene (ePE) 300, in accordance with some embodiments. In some embodiments, the process of FIG. 3 may be similar to that of FIG. 1. The method 300 can be implemented in a variety of contexts, including but not limited to medical devices, which may include implantable medical devices.

The method of processing ePE 300 may include heating an ePE substrate to a temperature above a melt temperature of ePE 310, cooling the ePE substrate 320, and forming the ePE substrate into an ePE article 330.

Heating the ePE substrate to the temperature above the melt temperature of ePE 310 is done with a heating source. The heating source may be similar to the heated component as described above with respect to FIG. 1. The heating source may be provided at various temperatures, including but not limited to above a melt temperature of ePE. For example, the temperature of the heating source may be provided between about 110° C. or about 180° C. In some embodiments, the heating source is provided at a temperature of from about 110° C. to about 120° C., from about 120° C. to about 130° C., from about 130° C. to about 140° C., from about 140° C. to about 150° C., from about 150° C. to about 160° C., from about 160° C. to about 170° C., and from about 170° C. to about 180° C. The heating source may include, but is not limited to, a press, a heated pad, an oven, or the like. In some embodiments, for example the heating source may be similar to the heated component 510, 610 as seen in FIGS. 5-6. In other embodiments, the heating source may be an indirect heating source like an environment set at a target temperature for heating the ePE substrate.

In some embodiments, heating the ePE substrate to the temperature above the melt temperature of ePE 310 may be done when the ePE substrate is unconstrained. When unconstrained, the ePE substrate may contract or collapse when heated. In other embodiments, the ePE substrate may be constrained in an X-direction (e.g., horizontally), a Y-direction (e.g., longitudinally), or in both the X-direction and the Y-direction. The ePE substrate may be constrained fully or partially in either of the X-direction and the Y-direction. When constrained, the ePE substrate may be limited or prevented from contracting or collapsing in the constrained direction. When heating the ePE substrate to the temperature above the melt temperature of ePE, the ePE substrate has a first size.

Referring still to FIG. 3, the method of processing ePE 300 further includes cooling the ePE substrate 320. Cooling the ePE substrate may be done by removing the ePE from the heating source. For example, after a specified amount of time, after a target ePE substrate temperature is reached, or when desired material properties are achieved, the ePE substrate may be removed from the heating source such that the ePE substrate cools. The ePE substrate may be cooled at room temperature, may be placed in an environment that is cooler than room temperature (e.g., a freezer), or may be slowly cooled in an environment with a temperature higher than room temperature. In some embodiments, the environment in which the ePE substrate is cooled may be at a stable temperature or may be a variable temperature.

In some embodiments, the variable temperature of the environment allows the ePE substrate to be cooled at a controlled rate. The rate of cooling of the ePE substrate may be constant or may be variable. Upon cooling, the ePE retracts or shrinks-back to a second size. The second size may be smaller than the first size. It is understood that once the ePE substrate is cooled such that the ePE is at or below a predetermined temperature, that the retraction of the ePE substrate ceases and the size of the ePE substrate is stable. The predetermined temperature may be the melt temperature of the ePE substrate. It is understood that during the cooling process, as the ePE substrate retracts, specific material properties may be imparted to the ePE substrate that may be desirable in certain contexts. For example, the ePE substrate that has been cooled and retracted may include selective expansion, better adhesion to secondary structures such as metals, PE or other polymers, stored length in the ePE substrate, distension when yielded, and so forth. In some embodiments, the predetermined temperature may be the melt temperature of the ePE substrate. The predetermined temperature may be at least partly dependent on the type of ePE used (e.g., e.g., expanded lower molecular weight polyethylene or expanded high molecular weight polyethylene). Once cooled, the ePE substrate is capable of expansion or distention at temperatures substantially below the melt temperature (e.g., room temperature). The ePE substrate may also still capable of expansion or distention at temperatures above the melt temperature.

Referring still to FIG. 3, after cooling the ePE substrate, the ePE substrate may be formed into an ePE article. In some embodiments, the second size of the ePE substrate may be formed into the ePE article. Forming the ePE substrate into the ePE article 330 may be done at an elevated temperature at or below the temperature of the heating source. In some embodiments, the ePE article may be formed into a tubular article (e.g., a tubular article 540 of FIG. 5), formed into a flat article (e.g., a flat article 620, 630, 640, or 650 of FIG. 6), formed into a film, or formed into a laminate made of multiple layers of processed ePE. Other articles may also be contemplated. In a medical application, the tubular article may be a graft and the flat article may be a hernia patch, a cardiovascular patch, a neuro membrane, and so forth.

In some embodiments, forming the ePE substrate into the ePE article 330 may further include wrapping the ePE substrate onto a mandrel (e.g., a mandrel 530 of FIG. 5). This may form the ePE substrate into the tubular article (e.g., the tubular article 540 of FIG. 5). Further to this embodiment, forming the ePE substrate into the ePE article 130 may further include bonding or attaching (e.g., bonding using adhesive, melt bonding, or mechanical attachment using a suture) the ePE substrate to itself. In one embodiment, the bonding or attaching of the ePE substrate to itself may be done along a longitudinal line (e.g., a longitudinal melt bond line 535 of FIG. 5). Other embodiments in which the bonding or attaching the ePE substrate to itself is done along a horizontal line, a diagonal line, or the like, are contemplated. Bonding or attaching the ePE substrate to itself may be done when the ePE is wrapped onto the mandrel.

Referring now to FIG. 4, a block diagram of a method of processing expanded polyethylene (ePE) 400 is provided, in accordance with some embodiments. In some embodiments, the process of FIG. 4 may be similar to that of FIG. 3. The method of processing ePE 400 can be implemented in a variety of contexts, including but not limited to medical devices, which may include implantable medical devices.

The method of processing ePE 400 includes a plurality of method steps. The method steps may include heating an ePE substrate to a temperature above the melt temperature of ePE, cooling the ePE substrate 420, forming the ePE substrate into an ePE article 430, and distending at least a portion of the ePE article 440.

The method step of heating the ePE substrate to the temperature above the melt temperature of ePE 410 may be substantially similar to the heating the ePE substrate to a temperature above a melt temperature of ePE 310 as described above with respect to FIG. 3. The heating source, for example, may be similar to the heating source as described above with respect to FIG. 3. The method step of cooling the ePE substrate 420 may be substantially similar to the cooling the ePE substrate 320 as described above with respect to FIG. 3. The method step of forming the ePE substrate into the ePE structure 430 may be substantially similar to the forming the ePE substrate into the ePE structure 330 as described above with respect to FIG. 3. Forming the ePE substrate into the ePE structure 430 may, for example, include forming the ePE structure 540 (see FIG. 5) and/or forming the ePE structure 620 (see FIG. 6).

In some embodiments, after retracting the ePE substrate, heat and/or pressure may be applied to the ePE substrate to limit the amount of distension that is possible. This allows the ePE substrate to have a locked-in or designated amount of possible distention. This may allow for control over the size of an ePE article formed from the ePE substrate.

The ePE structure is capable of distention. In some embodiments, distending at least a portion of the ePE structure 440 is performed on just a portion of the structure (e.g., at a specified longitudinal position). In some embodiments, the portion of the structure is at least one end of the ePE structure. In other embodiments, the portion of the structure is at a middle of the ePE structure between the ends. In other embodiments, distending at least a portion of the ePE structure 440 may be performed on the entire structure. In some embodiments, distending at least a portion of the ePE structure 440 may expand the ePE structure from the second size back to the first size. In some embodiments, distending at least a portion of the ePE structure 440 may expand the ePE structure to an intermediate size between the first size and the second size. In some embodiments, the intermediate size may include an intermediate diameter, for example an intermediate diameter DI in FIG. 7. In some embodiments, the intermediate size may also include an intermediate length, for example XI and YI in FIG. 8. In other embodiments, the intermediate size may include both the intermediate diameter and the intermediate length, for example the intermediate diameter DI and an intermediate length HI in FIG. 7.

In some embodiments, distending at least a portion of the ePE structure 440 includes radial expansion. In some embodiments, distending at least a portion of the ePE structure 440 includes one or both of longitudinal expansion and horizontal expansion. The longitudinal expansion may be in a Y-direction. The horizontal expansion may be in an X-direction. In some embodiments, distending at least a portion of the ePE structure 440 includes both radial expansion and one or both of longitudinal expansion and horizontal expansion. Other embodiments in which distending at least a portion of the ePE structure is done along in a diagonal direction or another non-straight direction are also contemplated.

In some embodiments, distending at least a portion of the ePE structure 440 may be done when the ePE structure is at room temperature (e.g., around 25° C.-30° C.). In some embodiments, distension can be done using a balloon (e.g., an angioplasty balloon). In some embodiments, the amount of distension of the ePE article is limited such that distention with the balloon is done to a predetermined ePE article size. In some embodiments, distending at least a portion of the ePE structure 440 is done by a manufacturer of the ePE structure. In some embodiments, distending at least a portion of the ePE structure 440 may be performed by a user or another party besides the manufacturer. In some embodiments, distending 240 the ePE structure is done by a physician prior to, during, or after a surgical procedure.

FIG. 5 illustrates an embodiment 500 in which a tubular article 540 is formed from an ePE substrate, in accordance with some embodiments. In some embodiments, the ePE substrate is an ePE sheet 515. The ePE sheet 515 may be similar to the ePE substrate as described above with respect to FIGS. 1-4. The ePE sheet 515 is provided as the starting material to form tubular article 540. In this embodiment, the tubular article 540 is the ePE structure as described above with respect to FIGS. 1-4. In some embodiments, the tubular article 540 is a graft. The method of forming the tubular article 540 may generally follow the method as described in FIGS. 1 and 2 and/or FIGS. 3 and 4.

The ePE sheet 515 is placed on the heated component 510 (e.g., a heated press). The heated component 510 is heated to a target temperature. For example, the target temperature of the heated component 510 may be provided between about 110° C. and about 180° C. In some embodiments, the heating source is provided at a temperature of from about 110° C. to about 120° C., from about 120° C. to about 130° C., from about 130° C. to about 140° C., from about 140° C. to about 150° C., from about 150° C. to about 160° C., from about 160° C. to about 170° C., and from about 170° C. to about 180° C. In this embodiment, the ePE sheet 515 is unconstrained on the heated component 510. In this embodiment, the ePE sheet 515 is approximately square shaped and defined by the first length L1.

The ePE sheet 515 is removed from the heated component 510. Upon removal from the heated component 510, the ePE sheet 515 is cooled as described with respect to removing the ePE substrate from the heated component 120, 220 and/or cooling the ePE substrate 320, 420. Upon cooling, the ePE sheet 515 retracts to a smaller ePE sheet 520. The smaller ePE sheet 520 is defined by the second length L2. In this embodiment, the second length L2 is smaller than the first length L1. In this embodiment, the smaller ePE sheet 520 retains the approximately square shape from the ePE sheet 515, but other configurations are contemplated where the smaller ePE sheet 520 retracts to an approximately rectangular shape or to a non-uniform shape in the X-direction (e.g., horizontally) and the Y-direction (e.g., longitudinally).

The smaller ePE sheet 520 is then formed into the tubular article 540. To form the tubular article 540, the smaller ePE sheet 520 is wrapped around a mandrel 530. In this embodiment, the mandrel 530 is tubular-shaped with a diameter M1. The smaller ePE sheet 520 wrapped around the mandrel 530 may be melt bonded to itself along a longitudinal line to create a longitudinal melt bond line 535. An excess of material 525 may be removed (e.g., cut away) from the smaller ePE sheet 520 to form the tubular article 540. In this embodiment, the tubular article 540 has a first diameter D1. The first diameter D1 may be substantially similar to the mandrel diameter M1.

The tubular article 540 is capable of distension to a distended tubular article 550. The tubular article 540 may be capable of distention when cooled. In this embodiment, the tubular article 540 is capable of distention in a radial direction after the retraction of the ePE sheet 515. The tubular article 540 may be radially distended using a balloon. In one embodiment, the distended tubular article 550 may only have a portion radially distended such that the distended tubular article 550 increases to a second diameter D2 at one end, where the second diameter D2 is larger than the first diameter D1. In another embodiment, a middle portion of the tubular article 540 may be radially distended. In other embodiments, the whole tubular article 540 may be radially distended. In still other embodiments, the tubular article 540 may be both radially distended and longitudinally distended.

FIG. 7 illustrates an embodiment in which the tubular article 540 of FIG. 5 is distended to an intermediate size, in accordance with some embodiments. FIG. 7 illustrates tubular article 540 which has the first diameter D1 and a first length H1.

Generally, the first diameter D1 and a first length H1 define the first size of the tubular article 540. The tubular article 540 may be radially distended to the distended tubular article 550 which has a second diameter D2 and longitudinally distended to a second length H2. Generally, the second diameter D2 and the second length H2 define the second size of the tubular article 540, or the distended tubular article 550. In some embodiments, the tubular article 540 may be distended to an intermediate tubular article 545 which has an intermediate size. The intermediate size is between the first size and the second size. In this embodiment, the tubular article 540 is distended in both the radial direction and the longitudinal direction. In other embodiments, the tubular article 540 may be distended in only one of the radial direction and the longitudinal direction.

In this embodiment, the intermediate size is defined by an intermediate diameter DI and an intermediate length HI. In some embodiments, the tubular article 540 may be distended from the first size to the intermediate size to create the intermediate tubular article 545 at a first temperature. The intermediate tubular article 545 may then be distended from the intermediate size to the second size to create the distended tubular article 550 at a second temperature. In some embodiments, the first temperature is higher than the second temperature. In some examples, the second temperature is at room temperature.

FIG. 6 illustrates an embodiment 600 in which a flat article 620 is formed from an ePE substrate, in accordance with some embodiments. In this embodiment, the ePE substrate is an ePE sheet 615. The ePE sheet may be similar to the ePE substrate as described above with respect to FIGS. 1-4. The ePE sheet 615 is provided as a starting material to form flat article 620. In this embodiment, the flat article 620 is the ePE structure as described with respect to FIGS. 1-4. In some embodiments, the flat article 620 may be a hernia patch, a cardiovascular patch, a neuro membrane, and so forth. The flat article may also be a film or a multi-layered laminate. The method of forming the flat article 620 may generally follow the method as described in FIGS. 1 and 2 and/or FIGS. 3 and 4.

The ePE sheet 615 may be placed on the heated component 610 (e.g., a t-shirt press), which may be similar to the heated component 510 (FIG. 5). The heated component 610 is heated to a target temperature. For example, the target temperature of the heated component may be provided between about 110° C. and about 180° C. In some embodiments, the heated component 610 is provided at a temperature of from about 110° C. to about 120° C., from about 120° C. to about 130° C., from about 130° C. to about 140° C., from about 140° C. to about 150° C., from about 150° C. to about 160° C., from about 160° C. to about 170° C., and from about 170° C. to about 180° C. In this embodiment, the ePE sheet 615 is unconstrained on the heated component 610. In other embodiments, the ePE sheet 615 may be constrained in the X-direction (e.g., horizontally), the Y-direction (e.g., longitudinally), or in both the X-direction and the Y-direction. In this embodiment, the ePE sheet 615 may be approximately rectangular-shaped and defined by a first X-dimension X1 and a first Y-dimension Y1. Other shapes for the ePE sheet 615, such as a square or an irregular shape are also contemplated.

The ePE sheet 615 is removed from the heated component 610. The ePE sheet 615 is cooled as described above with respect to removing the ePE substrate from the heated component 120, 220 and/or cooling the ePE substrate 320, 420. Upon removal from the heated component 610, the ePE sheet 615 retracts or shrinks to a smaller ePE sheet 620. The smaller ePE sheet 620 is defined by a second X-dimension X2 and a second Y-dimension Y2. In this embodiment, the smaller ePE sheet 620 retains the approximately rectangular shape from the ePE sheet 615, but other configurations are contemplated where the smaller ePE sheet 620 retracts to a non-uniform shape in the X-direction and the Y-direction. In one embodiment, the smaller ePE sheet 620 is the flat article 620.

The ePE substrate, or flat article 620, is formed by retracting (e.g., naturally) the ePE sheet 615 via a heating to cooling method as described above with respect to FIGS. 1-4. The flat article 620 is capable of distention when cooled. The flat article 620 is capable of longitudinal or horizontal distension after retraction of the ePE sheet 615. In one embodiment, the flat article 620 is capable of distension in both the X-direction (e.g., horizontally) and the Y-direction (e.g., longitudinally) to form an XY flat article 630. The XY flat article is defined by a third X-dimension X3 and a third Y-dimension Y3. In some embodiments, the third X-dimension X3 and the third Y-dimension Y3 are larger than the second X-dimension X2 and the second Y-dimension Y2 of the flat article 620, respectively. In some embodiments, the third X-dimension X3 and the third Y-dimension Y3 are smaller than the first X-dimension X1 and the first Y-dimension Y1 of the ePE sheet 615, respectively. In other embodiments, the third X-dimension X3 and the third Y-dimension Y3 are the same as the first X-dimension X1 and the first Y-dimension Y1 of the ePE sheet 615, respectively.

In some embodiments, the flat article 620 is capable of distension in just the Y-direction (e.g., longitudinally) to form a Y flat article 640. This may occur if the ePE sheet 615 is constrained either partially or fully in the X-direction (e.g., horizontally) during the heating to cooling method. The Y flat article 640 is defined by a fourth X-dimension X4 and a fourth Y-dimension Y4. In some embodiments, the fourth X-dimension X4 is the same as the second X-dimension X2 of the flat article 620. In some embodiments, the fourth Y-dimension Y4 is larger than the second Y-dimension Y2 of the flat article 620. In some embodiments, the fourth Y-dimension Y4 is smaller than the first Y-dimension Y1 of the ePE sheet 615. In other embodiments, the fourth Y-dimension Y4 is the same as the first Y-dimension Y1 of the ePE sheet 615.

In some embodiments, the flat article 620 is capable of distension in just the X-direction (e.g., horizontally) to form an X flat article 650. This may occur if the ePE sheet 615 is constrained either partially or fully in the Y-direction (e.g., longitudinally) during the heating to cooling process. The X flat article 650 is defined by a fifth X-dimension X5 and a fifth Y-dimension Y5. In some embodiments, the fifth Y-dimension Y5 is the same as the second Y-dimension Y2 of the flat article 620. In some embodiments, the fifth X-dimension X5 is larger than the second X-dimension X2 of the flat article 620. In some embodiments, the fifth X-dimension X5 is smaller than the first X-dimension X1 of the ePE sheet 615. In other embodiments, the fifth X-dimension X5 is the same as the first X-dimension X1 of the ePE sheet 615.

FIG. 8 illustrates an embodiment in which the flat article 620 of FIG. 6 is distended to an intermediate size, in accordance with some embodiments. FIG. 8 illustrates flat article 620 which has the second X-dimension X2 and the second Y-dimension Y2. Generally, the second X-dimension X2 and the second Y-dimension Y2 define a first size of flat article 620. The flat article may be horizontally and longitudinally distended to the XY flat article 630 which has the third X-dimension X3 and third Y-dimension Y3. Generally, the third X-dimension X3 and the third Y-dimension Y3 define a second size of flat article 620, for the XY flat article 630. In some embodiments, the flat article 620 may be distended to an intermediate flat article 625 which has an intermediate size in between the first size and the second size. In this embodiment, the intermediate size is defined by an intermediate X-dimension XI and an intermediate Y-dimension YI. In some embodiments, the flat article 620 may be distended from the first size to the intermediate size to create the intermediate flat article 625 at a first temperature. The intermediate flat article 625 may then be distended from the intermediate size to the second size to create the XY flat article 630 at a second temperature. In some embodiments, the first temperature is higher than the second temperature. In some examples, the second temperature is at room temperature. Although this embodiment is described above with respect to the XY flat article 630, a similar intermediate size may be formed prior to Y flat article 640 and X flat article 650.

The ePE substrate as described above with respect to FIGS. 1-8 may experience a change in its material properties during the heating to cooling method.

The ePE substrate, prior to processing, may exhibit high porosity, high surface area, and a high degree of crystallinity. In some embodiments, after the ePE substrate has been retracted via the heating to cooling method, the retracted ePE, or the ePE structure, may have a reduced porosity and a reduced surface area. In some embodiments, after the ePE substrate has been retracted via the heating to cooling process, the retracted ePE may have a similar porosity as prior to the heating to cooling process, wherein the pore size is smaller after retraction as compared to before retraction. However, the retracted ePE may not be reduced to a fully-densified state.

Instead, the retracted ePE may have pores with a smaller size than that of the starting material such that the ePE substrate retains its porosity throughout processing as described in FIGS. 1-4. In some embodiments, the smaller pore size may not be distorted in shape from that of the ePE substrate prior to processing. In other embodiments, the smaller pore sizes may be distorted in shape from that of the ePE substrate prior to processing. Further details on material property changes are discussed with respect to Examples 1 and 2.

Similarly, a microstructure of the ePE substrate may change upon retraction. The starting ePE substrate, prior to processing, may have a microstructure consisting essentially of fibrils of different lengths and nodes. In some embodiments, the fibrils may be serpentine fibrils. In some embodiments, the fibrils may be substantially all serpentine fibrils. In some embodiments, the retracted ePE may retain a similar microstructure to that of the ePE substrate prior to processing. However, in some embodiments, the fibrils of a smaller length may be lost upon processing and retraction of the ePE substrate. In some embodiments, upon retraction, the microstructure of the retracted ePE may reform, or create new connections. For example, new connections may be made fibril to fibril, fibril to node, or node to node.

These new connections may be made in any direction. The new connections may reduce the pore size of the retracted ePE substrate, but the retracted ePE substrate remains porous. When the retracted ePE substrate is then distended, the at least some of the new connections may be broken such that pore size increases. In some embodiments, when a laminate or another ePE article with layers of ePE is made, new connections in the microstructures can be made between the layers of ePE during retraction. Further details on microstructure and other structural changes are discussed with respect to Examples 1 and 2.

An expansion ratio between a precursor ePE, or the starting ePE material, and the retracted ePE substrate may be affected by the thermal and/or expansion history of the precursor ePE. For example, the ePE precursor may have been initially expanded with directionality (e.g., in the X-direction, Y-direction, Z-direction, and/or radial-direction, or any combination thereof) such that the ePE substrate retracts with the same directionality. This may occur due alignment of the fibrils of the microstructure of the precursor ePE where retraction aligns with the fibrils. Similarly, the ePE substrate may distend aligned with the fibrils. In this regard, to control the retraction and distention directionality of the ePE substrate, the precursor ePE microstructure fibril alignment may be controlled.

In other embodiments, the presence or absence of restraints in any of the X-direction, Y-direction, Z-direction, radial direction, or any combination therein may influence the directionality of retraction and distension of the ePE substrate. In some embodiments, the fibrils may align in the X-direction (e.g., horizontally) upon retraction such that the retracted ePE is only capable of retraction and distension in the X-direction. This may occur if the ePE substrate is constrained in the Y-direction (e.g., longitudinally) during the heating to cooling method. In other embodiments, the fibrils may align in the Y-direction upon retraction such that the retracted ePE is capable of retraction and distension in the Y-direction. This may occur if the ePE substrate is constrained in the X-direction during the heating to cooling process. Similarly, the ePE substrate can be constrained in the Z-direction (e.g., through the thickness) or constrained radially (e.g., with a mandrel) to influence the directionality of the fibrils. Further, the alignment of the retraction and distension may affect the direction in which the new connections in the microstructure are made in retraction and broken in distension. In this regard, to control the retraction and distention directionality of the ePE substrate, the ePE substrate can be constrained.

### EXAMPLES

### Example 1

In a first example, three ePE substrates were heated to temperatures above the melt temperature. A first ePE substrate 700 was heated to about 127° C., a second ePE substrate 702 was heated to about 130° C., and a third ePE substrate 704 was heated to about 133° C. Each of the first, second, and third ePE substrates 700, 702, 704 comprised a first, porous ePE film.

The first ePE substrate 700, the second ePE substrate 702, and the third ePE substrate 703 where each shaped as a tube and then heated. Heat was substantially uniformly applied to each of the first, second, and third ePE substrates 700, 702, 704 using a mandrel, though other heating sources may be used. When heating each of the first, second, and third ePE substrates 700, 702, 704, pressure was held constant without vacuum. Constant, low pressure of approximately 2 psi was applied using an overwrap.

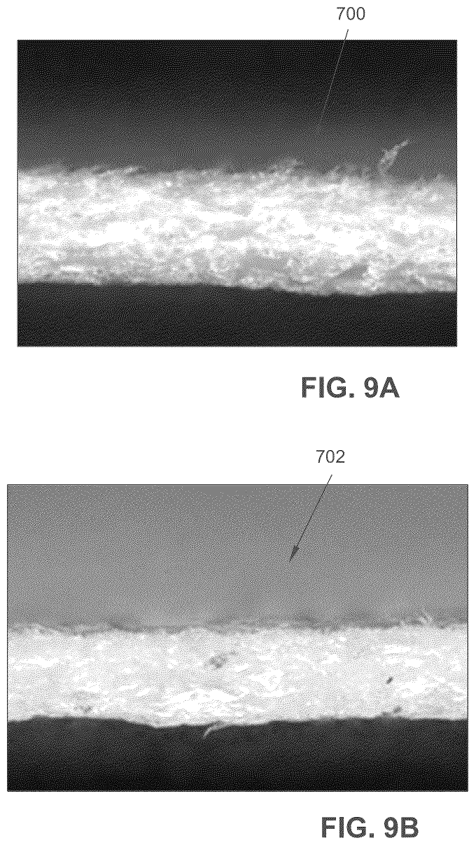

FIG. 9A shows the first ePE substrate 700 after being heated to 127° C. and then cooled. FIG. 9B shows the second ePE substrate 702 after being heated to 130° C. and then cooled. As observed, as process temperature is increased above the melt temperature, the ePE substrate melts such that the material contracts and densifies. As shown, the second ePE substrate 702 decreases in thickness, and is more compact, relative to the first ePE substrate 700. The second ePE substrate 702 also appears to tighten or densify such that the microstructure condenses. Additionally, the second ePE substrate 702 has less visible layering or less space within the ePE substrate, further showing a more densified, or more condensed material as compared to the first ePE substrate 700.

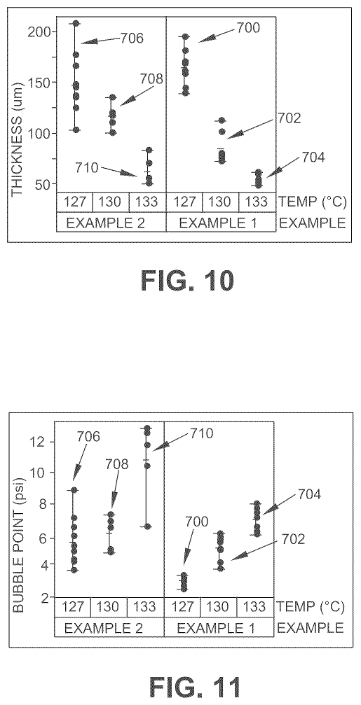

Turning to FIG. 10, the thickness of the first, second, and third ePE substrates 700, 702, 704 was measured in microns (μm) after the respective substrates were heated. As shown by the data, as processing temperature increases, thickness of the respective substrate decreases. In other words, a thickness of the third ePE substrate 704 is smaller than a thickness of the second ePE substrate 702, and the thickness of the second ePE substrate 702 is smaller than a thickness the first ePE substrate 700. As described with respect to FIGS. 9A-9B, the decrease in thickness may be correlated to densification and contraction of the ePE substrate and/or condensing of the microstructure of the ePE substrate.

Turning to FIG. 11, the bubble point of the first, second, and third ePE substrates 700, 702, 704 were measured in psi. As shown by the data, as processing temperature increases above the melt temperature, the bubble point of the respective substrate increases. The bubble point may be correlated to a pore size present in the ePE substrate. As bubble point increases, it indicates that the pore size of the substrate decreases. In other words, a pore size of the third ePE substrate 704 is smaller than a pore size of the second ePE substrate 702, and the pore size of the second ePE substrate 702 is smaller than a pore size of the first ePE substrate 700. As described with respect to FIGS. 9A-9B, the increase in bubble point may also be correlated to densification and contraction of the ePE substrate and/or condensing of the microstructure of the ePE substrate.

Additionally, the pore size may correspond to an ability of the article to selectively allow or reduce cellular ingress, ingrowth, and/or attachment within its structure. A smaller pore size may allow the respective article to reduce or limit cellular ingress therethrough, which may be desirable in some applications, including but not limited to aortic devices. A larger pore size may allow the respective article to allow cellular ingress therethrough. As such, processing temperature may be selected to increase or decrease pore sizes as desired, to either allow or reduce cellular ingrowth, respectively.

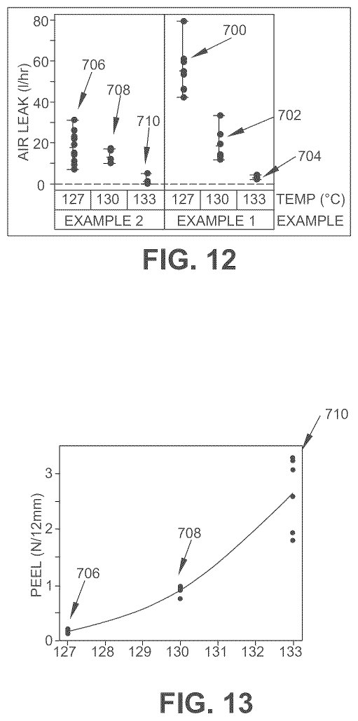

Turning to FIG. 12, airflow, or air leak through the ePE substrate was measured for the first, second, and third ePE substrates 700, 702, 704 in liters per hour (l/hr). The airflow measurement was done using leak detection equipment from ATEQ®. As processing temperature was increased, the air leak of the respective substrate decreased. The air leak volume may be correlated to a pore size present in the ePE substrate as large pore size would allow more air to leak through the ePE substrate. This indicates that the pore size of the respective substrates decreased as processing temperature was increased. In other words, a pore size of the third ePE substrate 704 is smaller than a pore size of the second ePE substrate 702, and the pore size of the second ePE substrate 702 is smaller than a pore size of the first ePE substrate 700. As described with respect to FIGS. 9A-9B, the decrease in air leak may be correlated to densification and contraction of the ePE substrate and/or condensing of the microstructure of the ePE substrate.

Though the above example were described with respect to tubular shaped ePE substrates, flat ePE substrates, or other shapes of ePE substrates, may show similar behavior, and similar material property changes, upon being heated to a temperature above the melt.

### Example 2

In a second example, three ePE substrates were heated to temperatures above the melt. A fourth ePE substrate 706 was heated to about 127° C., a fifth ePE substrate 708 was heated to about 130° C., and a sixth ePE substrate 710 was heated to about 133° C. Each of the fourth, fifth, and sixth ePE substrates 706, 708, 710 comprised a second, porous ePE film, which was different than the first porous ePE film of Example 1.

Similar to Example 1, the fourth ePE substrate 706, the fifth ePE substrate 708, and the sixth ePE substrate 710 were each shaped as a tube prior to heating. Heat was substantially uniformly applied to each of the fourth, fifth, and sixth ePE substrates 706, 708, 710 using a mandrel, though other heating sources may be used. When heating each of the fourth, fifth, and sixth ePE substrates 706, 708, 710, pressure was held constant without vacuum. Constant, low pressure of approximately 2 psi was applied using an overwrap.

Similar to Example 1, the thickness, bubble point, and air leak were measured for each of the fourth, fifth, and sixth ePE substrates 706, 708, 710. The trends of the material properties were similar to those found in Example 1. As shown in FIG. 10, as processing temperature was increased above the melt temperature, thickness of the respective ePE substrate decreased. As shown in FIG. 11, as processing temperature was increased above the melt temperature, bubble point increased. As shown in FIG. 12, as processing temperature was increased above the melt temperature, the air leak of the respective substrate decreased. These results indicate that increasing processing temperature may be correlated to densification and contraction of the ePE substrate, condensing of the microstructure of the ePE substrate, and/or a decrease in pore size of the ePE substrate. This also indicates that densification, condensing of the microstructure, and decreased pore size of the respective ePE substrates upon increasing processing temperature is not limited to just one type of porous ePE film, but may be observed with both the first and second porous ePE films.

Turning to FIG. 13, a peel strength was measured for each of the fourth, fifth, and sixth ePE substrates 706, 708, 710. The peel strength was measured as force to peel the substrate back about 12 mm and is shown in units of N/12 mm. As shown by the data, as processing temperature increased above the melt temperature, the peel strength of the respective substrate was increased. In other words, a force required to pull the third ePE substrate 704 is larger than a force required to pull the second ePE substrate 702, and the force required to pull the second ePE substrate 702 is larger than a force required to pull the first ePE substrate 700. The increase in force may also be correlated to densification and compaction of the ePE substrate and/or condensing of the microstructure of the ePE substrate. The increased force needed to pull back the substrate indicates that the layers or space within the ePE substrate is decreased as the processing temperature increases, and new bonds may be made within the ePE substrate.

Though the above example was described with respect to tubular ePE substrates, flat ePE substrates, or other shapes of ePE substrates, may show similar behavior, and similar material property changes, upon being heated to a temperature above the melt.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

1. A method of processing expanded polyethylene (ePE) comprising:
positioning an ePE substrate onto a heated component, the ePE substrate having a first size;
heating the ePE substrate on the heated component;
removing the ePE substrate from the heated component such that the ePE substrate cools and retracts to a second size, wherein the second size is smaller than the first size; and
forming the ePE substrate into an ePE article.

2. The method of claim 1, wherein forming the ePE substrate into the ePE article includes wrapping the ePE substrate onto a mandrel.

3. The method of claim 2, wherein forming the ePE substrate into the ePE article includes melt bonding the ePE substrate to itself along a longitudinal line.

4. The method of claim 1, wherein positioning the ePE substrate onto the heated component includes providing the ePE substrate as a sheet of ePE.

5. The method of claim 1, wherein heating the ePE substrate includes heating the heated component between about 110 degrees Celsius and 180 degrees Celsius.

6. The method of claim 1, further comprising adhering the ePE article to a metal.

7. The method of claim 1, further comprising adhering the ePE article to another polyethylene structure.

8. The method of claim 1, further comprising distending at least a portion of the ePE article.

9. The method of claim 8, wherein distending at least a portion of the ePE article includes radial expansion.

10. The method of claim 8, wherein distending at least a portion of the ePE article includes longitudinal expansion.

11. A method of processing expanded polyethylene (ePE) comprising:
heating an ePE substrate to a temperature above a melt temperature of ePE, the ePE substrate having a first size;
cooling the ePE substrate, the ePE substrate retracting to a second size upon cooling, the second size being smaller than the first size; and
forming the ePE substrate into an ePE article.

12. The method of claim 11, wherein the ePE substrate is heated between about 110 degrees Celsius and 180 degrees Celsius.

13. The method of claim 11, further comprising the step of distending at least a portion of the ePE article.

14. The method of claim 13, wherein distending at least a portion of the ePE article is done at a room temperature.

15. An ePE article made of expanded polyethylene (ePE) comprising:
an ePE substrate having been formed into an ePE article, the ePE article being formed by retracting the ePE sheet via a heating to cooling process, the ePE article being capable of distention.

16. The ePE article of claim 15, wherein the ePE substrate is formed into a graft.

17. The ePE article of claim 15, wherein the ePE substrate is formed into the ePE article using a mandrel.

18. The ePE article of claim 15, wherein the ePE article is capable of distention in a longitudinal direction.

19. The ePE article of claim 15, wherein the ePE article is capable of distention in a radial direction.

20. The ePE article of claim 15, wherein the ePE article is porous.
